(19) United States
(12) Patent Application Publication
(10) Pub. No.: US 2014/0000615 A1
(43) Pub. Date: Jan. 2, 2014

(54) SYSTEM FOR REDUCTION OF AIR LEAKS WITH USE OF CPAP MASKS

(71) Applicant: Alan Wanderer, Bozeman, MT (US)

(72) Inventor: Alan Wanderer, Bozeman, MT (US)

(21) Appl. No.: 13/784,907

(22) Filed: Mar. 5, 2013

Related U.S. Application Data
(60) Provisional application No. 61/695,430, filed on Aug. 31, 2012.
(60) Provisional application No. 61/664,787, filed on Jun. 27, 2012.

(51) Int. Cl. A61M 16/06; A61M 16/00

(52) U.S. Cl. CPC A61M 16/0683; A61M 16/0057
USPC 128/205.25; 128/206.24

(57) ABSTRACT

A system of the invention is a housing-liner apparatus complex and/or eye shield comprising an air leak preventing system is provided for use with continuous positive airway pressure devices, e.g. CPAP mask. The system includes a housing-liner and/or an eye shield reversibly or irreversibly attached to a mask, and adjustable straps extending from the housing-liner and/or eye shield to attach onto a mask to create an improved seal between the mask and the user. Beneficially, the system muffles noise associated with air leakage, and reduces other side effects with CPAP masks, e.g. slippage of the lower part of the mask into the user's mouth and/or puffing out of cheeks during sleep. Furthermore, the system may include a chin support to reduce mouth opening of the user during sleep. Overall, the system creates greater user comfort by maintaining the interface between the mask and user's face during sleep.

### CROSS-REFERENCE TO RELATED APPLICATIONS

This application claims priority under 35 U.S.C. §119 to provisional applications U.S. Ser. No. 61/695,430 filed Aug. 31, 2012 and U.S. Ser. No. 61/664,787 filed Jun. 27, 2012, herein incorporated by reference in its entirety.

### FIELD OF THE INVENTION

The present invention relates generally to the field of medical devices. More specifically, but not exclusively, the invention relates to continuous positive airway pressure devices, and covers or liners therefore, in particular systems for reduction of air leaks, improving a device connection or contact with a user's face, maintaining air pressure within the device and/or improving patient comfort. Methods of achieving the same improvements for use of continuous positive airway pressure devices are further provided.

### BACKGROUND OF THE INVENTION

Continuous positive airway pressure (CPAP) is the use of positive pressure to maintain a continuous level of positive airway pressure to prevent collapse of airways in users prone to sleep apnea. CPAP at home utilizes machines specifically designed to deliver a constant or variable flow or pressure.

CPAP treatment involves a CPAP machine, with an air compressor that is a pressure flow generating device, a mask or other device that fits over a user's nose or nose and mouth (straps keep the mask in place while worn) and a tube that connects the mask to the CPAP machine. When the CPAP is turned on and the mask is in place on the user, air flows under pressure through the tube into the mask, allowing air to flow under pressure into the patient's airway. The pressured airflow is designed to reduce or eliminate airway collapse often occurring in the upper airway, such that air flow will be unimpeded into the user's lungs allowing for oxygen transport into the user's circulation and removal of carbon dioxide through exhalation ports of the mask. Some CPAP machines have other features as well, such as heated humidifiers. CPAP machines come in a variety of shapes and sizes, with the majority being small and lightweight.

The CPAP masks are typically a nasal or nasal/oral mask which fit on the face of a patient to interface the ventilator or pressure support system with the airway of the patient so that a flow of breathing gas can be delivered from the pressure flow generating device to the airway of the patient. Straps on the mask are designed to maintain such masks on the face of a patient to ensure best performance. In particular, there is headgear having upper and lower straps, each having opposite ends threaded through connecting elements provided on the opposite lower sides and top of a mask. Typically, patient interface devices include a mask shell having a cushion attached to the shell that contacts the surface of the patient. The mask shell and cushion are held in place by the headgear with straps that wrap around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat sleep apnea, the patient normally wears the patient interface device all night long while he or she sleeps. One concern in such a situation is that the patient interface device is as comfortable as possible; otherwise, the patient may avoid wearing the interface device, defeating the purpose of the prescribed pressure support therapy. It is also important that the interface device provide a tight enough seal against a patient's face without discomfort. A problem arises when the mask may be compressed against the patient's face in order for the mask to maintain a seal without any undue gas leaks around the periphery of the mask. This can cause facial sores and unsightly pressure marks upon arising after sleeping with the mask.

There is also a serious compliance problem for users of CPAP masks that leak when the user moves around in sleep, and especially when high pressures are required to maintain patency of the oral-pharyngeal airway and/or nasal airways. A further problem arises from the high pitched noise emitted from the masks due to leaking air which can awaken the user and/or sleep mate. Still other issues include awakening of a user by sudden puffing out of the user's cheeks while wearing a mask. This may be caused by excessive air pressure retained within the user's airway that seeks relief by expanding out the flexible cheeks of the user. Another problem that can occur during sleep with conventional CPAP full face masks, is the gradual slippage of the inferior border of the mask upwards into the oral cavity of the user, which can awaken the user and also cause air leak from the normally closed full face pressure system. This problem is exacerbated by the puffing of the cheeks which stretch the lower headgear straps causing the mask to move superiorly on the user's face, and allow the inferior pole of the mask to slip into the user's mouth. Other issues include mouth opening tendency when wearing a face mask that can cause dryness in the mouth when wearing a full face mask and/or break a tight pressure seal for nasal mask users. Opening the mouth also allows the lower jaw to fall posteriorly and/or inferiorly while sleeping, thereby enhancing posterior movement of the tongue toward the posterior pharynx and more airway obstruction.

The CPAP masks existing in the art have substantial leakage of air and/or oxygen. There is a significant need to reduce leaks and thereby maintain the patency of the user's upper airway. The present invention demonstrates reduction in air and/or oxygen leakage, which may cause noisy air leaks, thereby allowing the user to sleep more soundly. In addition creating a tighter seal around the mask and user's face may allow the sleep specialist to better titrate the pressure required to reduce apnea events and/or oxygen deprivation. The need for such reduction of leakage indicates a clear benefit to the device and methods of the invention that provide improved connection between the user's face and the CPAP mask.

It is clear there exists a need in the art for a device and method of use that ensures that the mask maintains connection between face and mask without interfering in flow of air/oxygen and/or vents from mask and allows more efficiency for air pressure delivery by reducing leaks, reduce the cheek puffing especially at high air pressures, and reducing slippage of masks upwards into the user's mouth. There is also a need in the art for a device and method of use that provides comfort for user to wear the mask, muffles and minimizes air leaking from the mask.

It is therefore a primary object, feature, and/or advantage of the present invention to overcome deficiencies in the art.

It is another object, feature, and/or advantage of the present invention to provide a mask with housing-liner and method of use that aids in connecting the face of a user with the mask without interfering in the flow of air and/or oxygen to user.

It is another object, feature, and/or advantage of the present invention to provide a mask with housing-liner and method of use that provides comfort when user wears the mask.

It is still another object, feature, and/or advantage of the present invention to provide a mask with housing-liner and method of use that is universal in that it can be altered structurally (e.g. dimensions, shape, etc.) with the same functional elements to fit virtually any type of CPAP unit.

It is a further object, feature, and/or advantage of the present invention to provide a mask with housing-liner and method of use that is easily added to any type of CPAP mask.

It is still a further object, feature, and/or advantage of the present invention to provide a mask with housing-liner and method of use that can be customizable, be cleaned, and can be easily adjustable.

It is another object, feature, and/or advantage of the present invention to provide a mask with housing-liner that will reduce air leakage.

It is yet another object, feature, and/or advantage of the present invention to provide a mask with housing-liner that allows for muffling of air leaks and overall provide more peaceful sleep for a user and a user's sleep mate.

Another object is to provide a mask with housing-liner that reduces puffing out of user's cheeks.

It is also an object of the present invention to provide a mask with housing-liner that prevents slippage of the inferior pole of the mask upwards into the user's mouth.

Yet still another object of the invention is to provide a chin support integrated as part of the housing-liner, such that a separate chin supporting strap is unnecessary.

These and/or other objects, features, and advantages of the present invention will be apparent to those skilled in the art. The present invention is not to be limited to or by these objects, features and advantages. No single embodiment need provide each and every object, feature, element or advantage.

### SUMMARY OF THE INVENTION

The present invention is a housing-liner that is an accessory for CPAP machines, including facemasks and/or nose masks of all sizes used with CPAP machines. The invention demonstrates reduced noise from air leakage which improves compliance for users and reduces the adverse side effects associated with sleep apnea patients who do not like using CPAP equipment.

The invention discloses a housing-liner apparatus. In another aspect, the invention discloses a system for reducing air leaks from an airflow mask. In a still further aspect, the invention discloses a method of improving the effectiveness of an airflow mask including reducing air leakage and/or noise associated with air leakage.

### BRIEF DESCRIPTION OF THE DRAWINGS

FIG. 1 is a perspective view of a type of commercially-available CPAP mask by itself (e.g. without the housing-liner according to the invention).

FIG. 2 is a perspective view of the same of CPAP mask in FIG. 1 positioned on a person.

FIG. 3 is a perspective view of a different type of commercially-available CPAP mask with a top post for upper headgear straps by itself (e.g. without the housing-liner according to the invention).

FIG. 4 is a perspective view of a commercially-available nasal type of CPAP on a patient by itself (e.g. without the housing-liner according to the invention).

FIG. 5 is an open display of a housing-liner suitable for use with the particular CPAP mask in FIG. 1. However, one skilled in the art will appreciate that modifications, including structural modifications, to the systems of the invention will ensure that the systems could be used for any type of CPAP mask according to embodiments of the invention.

FIG. 6 is a housing-liner according to an embodiment of the invention, shown as partially attached to the CPAP mask of FIG. 1.

FIG. 7 shows a further embodiment for use either alone and/or in combination with the housing-liner according to embodiments of the invention, including an eye shield liner for the upper part of the CPAP mask in FIG. 1 for shielding eyes from air leaks.

FIG. 8 shows the eye shield liner of FIG. 7 attached to mask of FIG. 1.

FIG. 9 shows the mask of FIG. 1 attached to a user, having housing-liner and an eye shield liner according to the invention attached to the mask, demonstrating a first step of attaching the housing-liner.

FIG. 10 is housing-liner and eye shield liner attached to mask in FIG. 1, demonstrating a second step of attaching the housing-liner with the lower straps of housing-liner wrapped around the lower regions of user's head.

FIG. 11 shows the lower straps of the housing-liner wrapped around the posterior head of a user according to an embodiment of the invention.

FIG. 12 shows the partially-attached housing-liner with the chin straps of housing-liner being the final step for attaching the housing-liner to a user's head.

FIG. 13 shows the chin straps pulled up onto side of user's head, demonstrating the final or third step of attaching the housing-liner according to embodiments of the invention.

FIGS. 14A-C and 15A-C represent a sleep record showing air leak using the mask in FIG. 1 without (FIGS. 14A-C) and with (FIGS. 15A-C) the attached housing-liner and eye shield liner respectively, according to the methods of the invention.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE PREFERRED EMBODIMENTS

### Description of Prior Art CPAP Masks Suitable for Use With the Housing-Liner

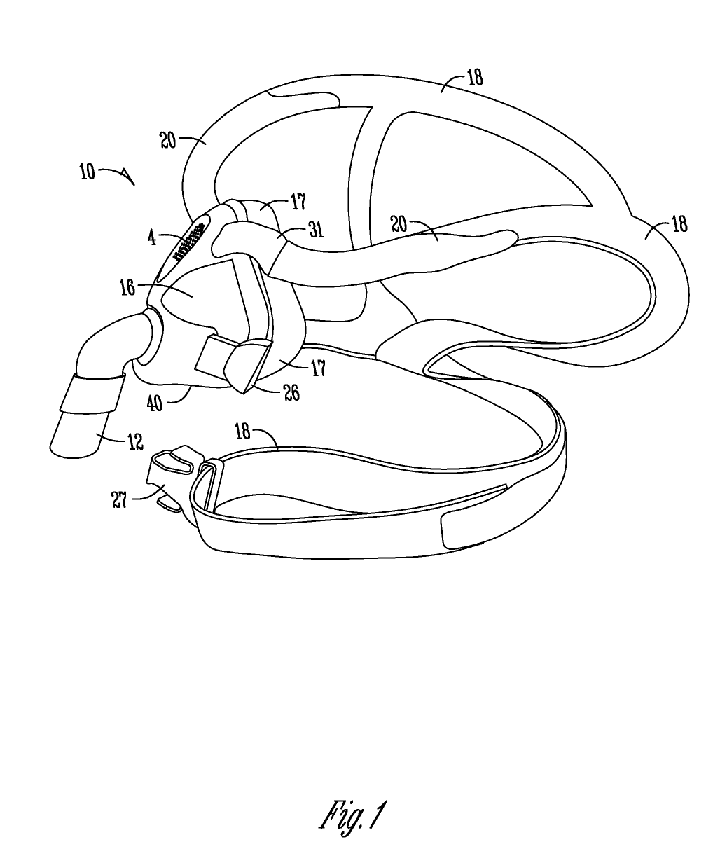

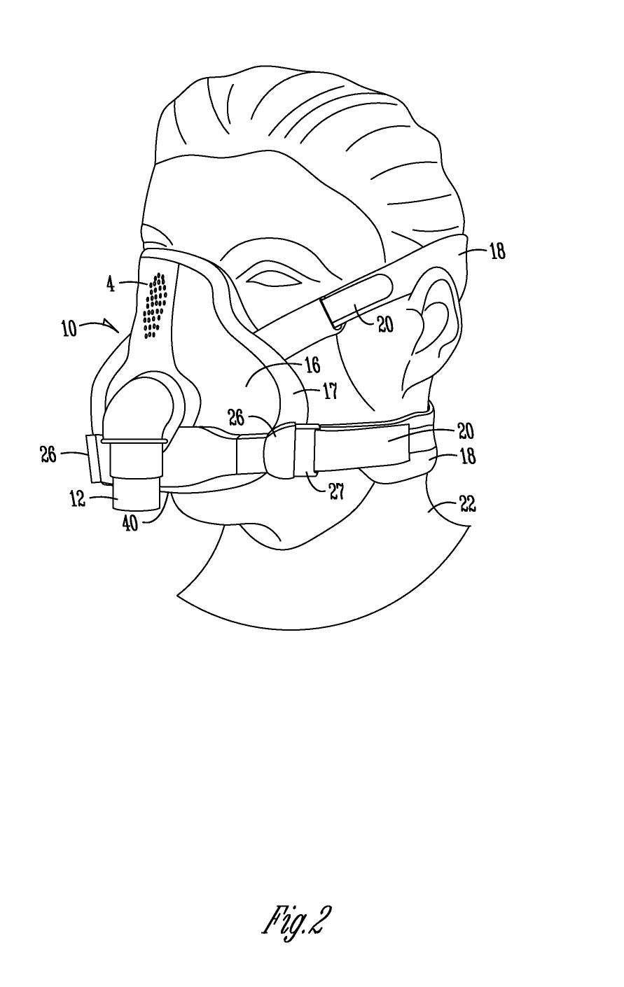

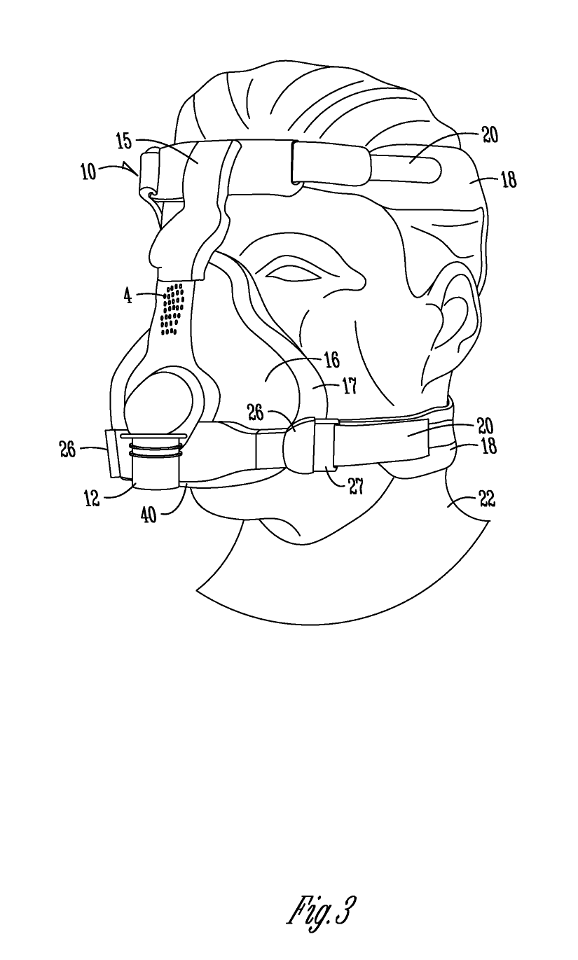

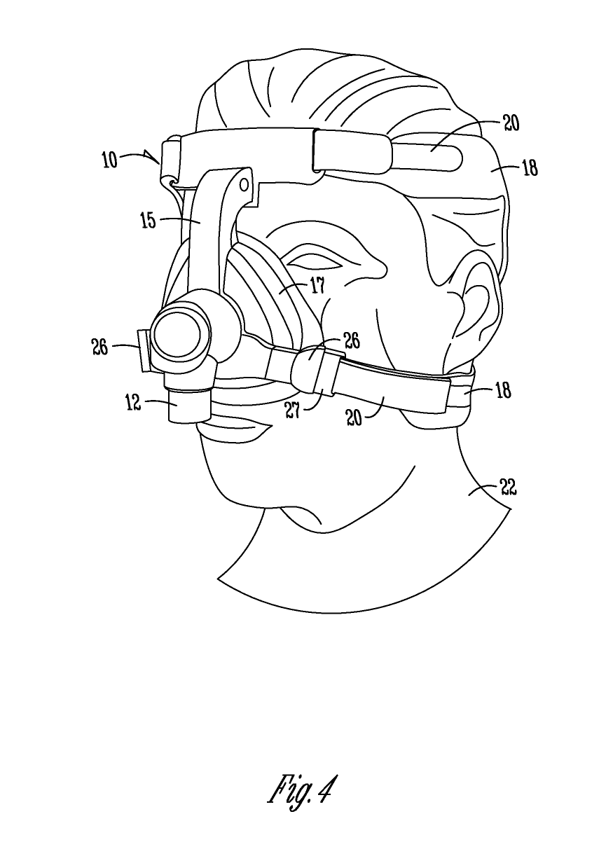

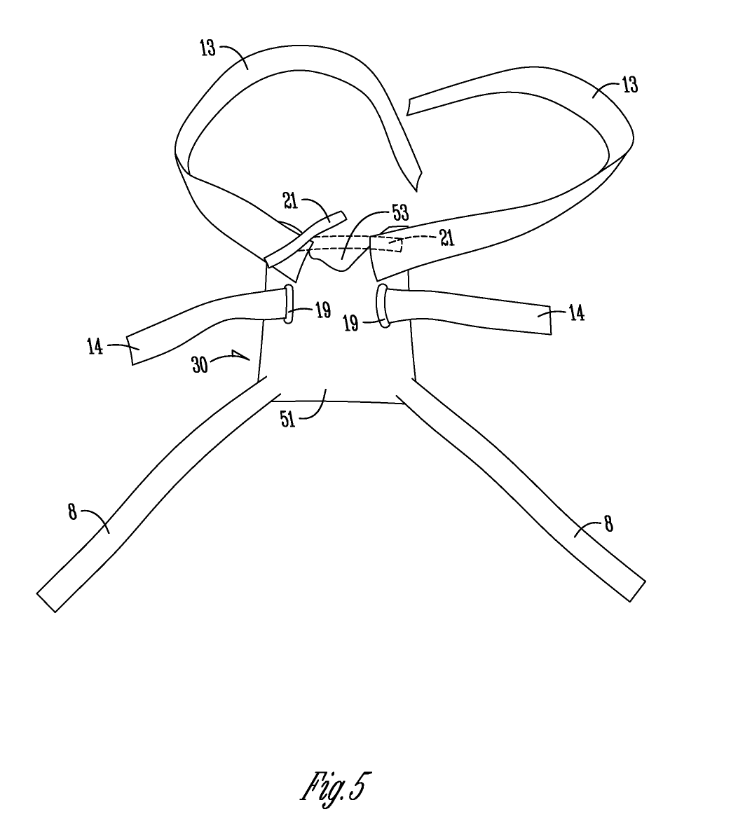

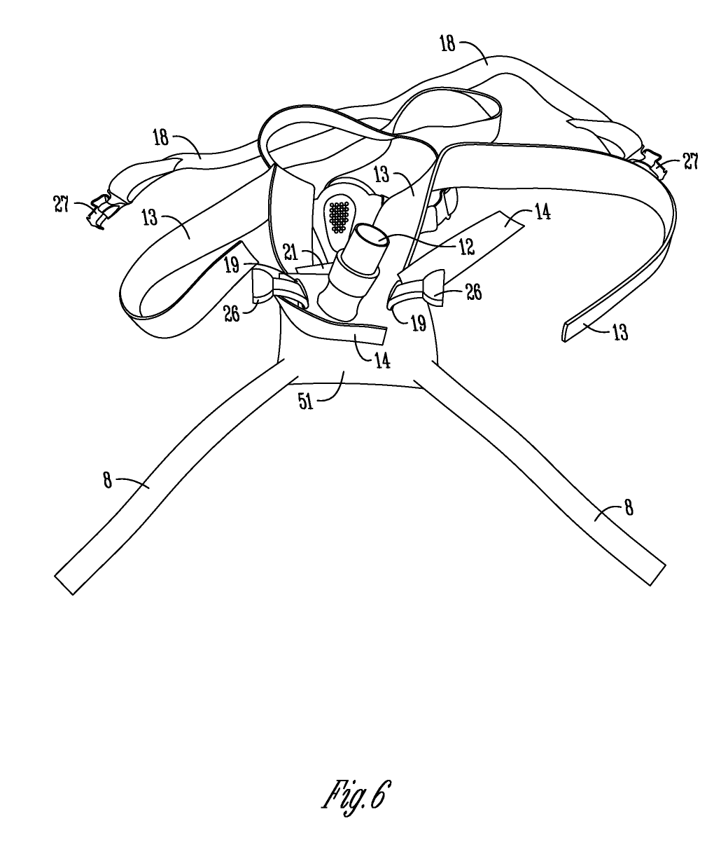

FIGS. 1 and 2 are perspective views of the same continuous positive airway pressure (CPAP) mask, shown individually positioned on the face of a user. The depicted CPAP mask is an exemplary CPAP mask commercially available at the time of the present invention. As noted, a CPAP mask is used by people having breathing conditions, such as sleep apnea or the like. The CPAP mask and air compressor provides continuous air pressure to the airway of the individual, thus preventing the airway from collapsing and blocking breathing of the individual. The CPAP mask 10 of FIGS. 1 and 2 is a mask covering generally the mouth and nose of a person 22. However, as is known in the art, CPAP masks may also cover just the nose of a person as seen in FIG. 4, just the mouth, or a combination of both the nose and mouth, FIG. 3. Any such styles of CPAP masks are suitable for use according to the apparatuses and methods of the present invention.

The CPAP mask 10 of FIGS. 1-3 include a mask section having an outer edge 16 that is shaped to surround a mouth and nose of the person 22. Generally, the mask 10 also includes a cushion section 17 positioned between the outer edge of the mask 10 and the face of the individual. The cushion portion 17 provides comfort and is intended to provide a seal between the mask 10 and the person 22 such that air is to not be leaked between the mask and person. The mask 10 also includes a tube or hose connection 12, which connects to a machine for providing the continuous air pressure. Openings 4 positioned on/within the mask 10 serve as portals for externalization of pressurized air and carbon dioxide emanating from the user.

In various embodiments of commercial CPAP masks it is contemplated that masks may be designed with support straps 18. The straps 18 are generally positioned around regions of the user's head, which may be adjusted and/or designed in a variety of ways, and can be adjusted via adjustable portions 20 to ensure a tight fit on the person. The adjustable portions 20 may include any known means of tightening or otherwise adjusting a strap, various examples of which are depicted in the figures to illustrate one or more types of adjustable portions which are connected to the support straps 18 of a CPAP mask.

As shown in the exemplary figures depicting commercially-available CPAP masks, there are two male means 27 (see e.g. FIGS. 1-3) attached to the lower head straps that lock the lower headgear straps 18 into the female attaching means 26 on the mask 10. The female attaching means 26 permit the strap 18 to integrate and fasten into both lower sides of the mask. As disclosed herein the attaching means may include various known mechanisms, not limited to those depicted in the figures, which are non-limiting examples.

A difference between CPAP masks in FIGS. 3, 4 from FIGS. 1, 2 is a vertical support strip 15 of the mask 10 that connects the mask to the upper restraining straps 18 that fasten around the upper region of the user's head.

While CPAP masks, such as those shown in FIGS. 1-4 are designed to fit tightly against a person's face, air may still escape through leaks between the mask (namely the cushion portion 17) and the person's face. The leaks may be caused by different facial shapes and sizes, faulty application or positioning of the mask onto a user, faulty designs of the mask, by movement of the user while sleeping, and/or as a result of a variety of other reasons.

### Housing-Liner Systems According to the Invention and Applications of Use Thereof

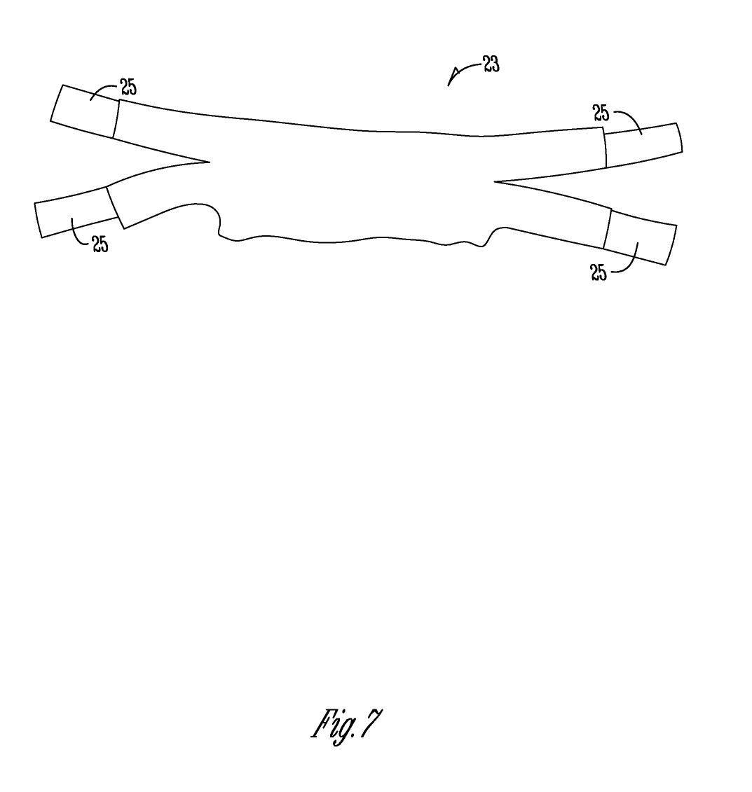

As a result of the various deficiencies of the CPAP masks commercially-available, the present invention, a housing-liner 30 as shown in FIG. 5 and further described herein is provided with a CPAP mask 10 in order to seal leaks between the mask and the user's face to improve the efficiency and comfort of the CPAP machine. The housing-liner 30 at least partially covers (e.g. may also nearly entirely cover and/or entirely cover) the CPAP mask 10 to reduce leaking from the interface of the cushion 17 and the user's face. The housing-liner 30 will also muffle noises that may occur due to leaking air from the interface of the mask with the face of the user. The housing-liner 30 has been described in prior embodiments in the preceding provisional applications as a mask housing-liner hybrid, and herein it is referred to as a housing-liner.

The housing-liner 30 can be made of any number of materials, such as for example, stretchable and/or breathable materials. The housing-liner 30 further includes attaching means on these stretchable and/or breathable materials to adjust the fitting of the housing-liner 30 over a CPAP mask. The attaching means may include for example, hooks and/or loops using Velcro attachments, elastic polyesters, thermoplastic elastomer and other compressible molding materials, without limiting the scope of the present invention. In an aspect, the materials are preferably washable, not subject to shrinkage, and may include a variety of designs in composition. The systems may further include various aesthetic modifications that are within the scope of the invention. Thus, colors, designs, logos, and the like are all contemplated to be part of the invention and may be included on the material of the housing-liner and/or systems per a user's desire. These and other variations of the embodiments of the invention are included within the scope of the invention.

A preferred housing-liner 30 is shown in FIG. 5. The material used for the housing-liner is depicted as a preferably stretchable, breathable, and thinly-milled material with one side comprised of Velcro loop and the other side being a smooth surface. The central body 51 of the housing-liner 30 serves as a chin support and emanating from it are three pair of straps (8, 13, 14) and a single strap 21. As shown in FIG. 5, the single strap 21 in a closed or affixed position (e.g. shown as a dashed line) creates a surrounded area 53 wherein the tube or hose connection 12 of the mask is disposed there through. As depicted in the various additional figures, the single strap 21 wraps over the tube or hose connection 12 of the mask provide yet a further point of connection for maintaining a fixed position between the housing-liner and the mask to ensure best fit and wear of the system according to the invention.

In an aspect, there are two identical holes 19 at the junction of straps 13 and 14 or at the junction of straps 14 and the body of the housing liner (as shown in FIG. 5). The holes 19 are one embodiment of an attachment means for the housing-liner according to an embodiment of the invention wherein a mask having attachment means 26, 27 is employed. However, one skilled in the art will ascertain that additional attachment means may be utilized in place of the holes 19, such as for example at least one strap and/or a set of straps, clips, Velcro, loops, ties, etc.

Another set of two identical holes (not shown) may be located in the central body 51 of the housing-liner. These holes will superimpose over two covered ports (not shown) in the mask 10 of FIG. 1 which are designated for oxygen lines to enter through the CPAP mask, if needed. As one skilled in the art will ascertain, the placement of the holes for entry of oxygen lines through the housing-liner and mask can be placed at any location on the central body 51 of the housing-liner 30 to correspond with the placement of the covered ports on a CPAP mask.

The steps for attaching the housing-liner 30 depicted in FIG. 5 according to embodiments of the invention are set forth in a stepwise fashion to illustrate how a user would employ the housing-liner. Of course one skilled in the art, based on the disclosure of the present invention, will ascertain additional means of attaching or affixing the housing-liners to a CPAP mask. Such methods and applications are included within the scope of the present invention.

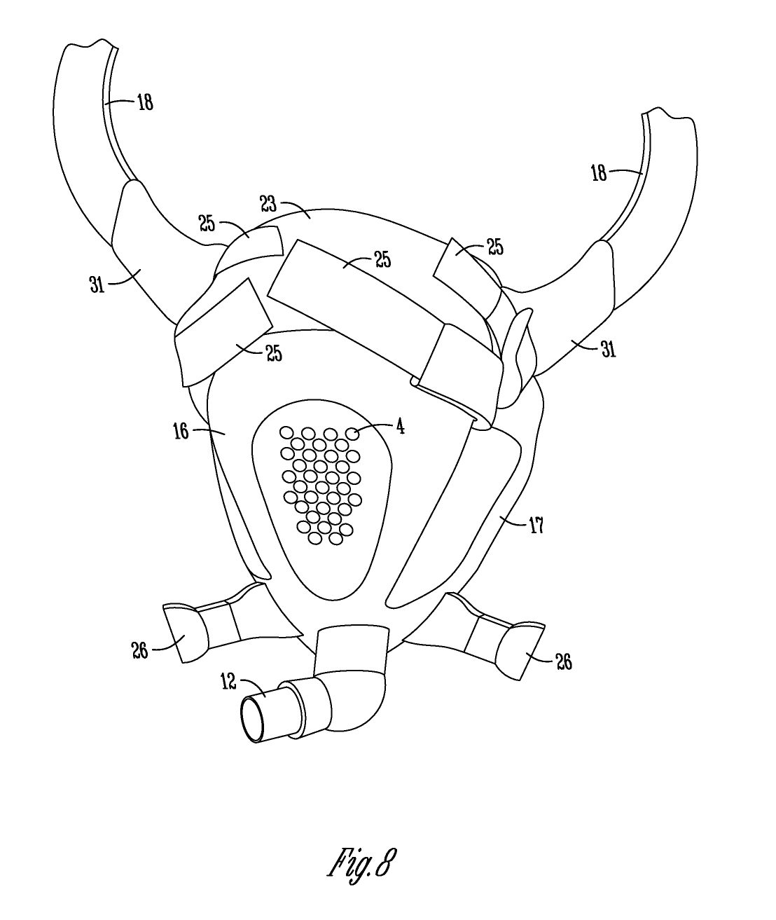

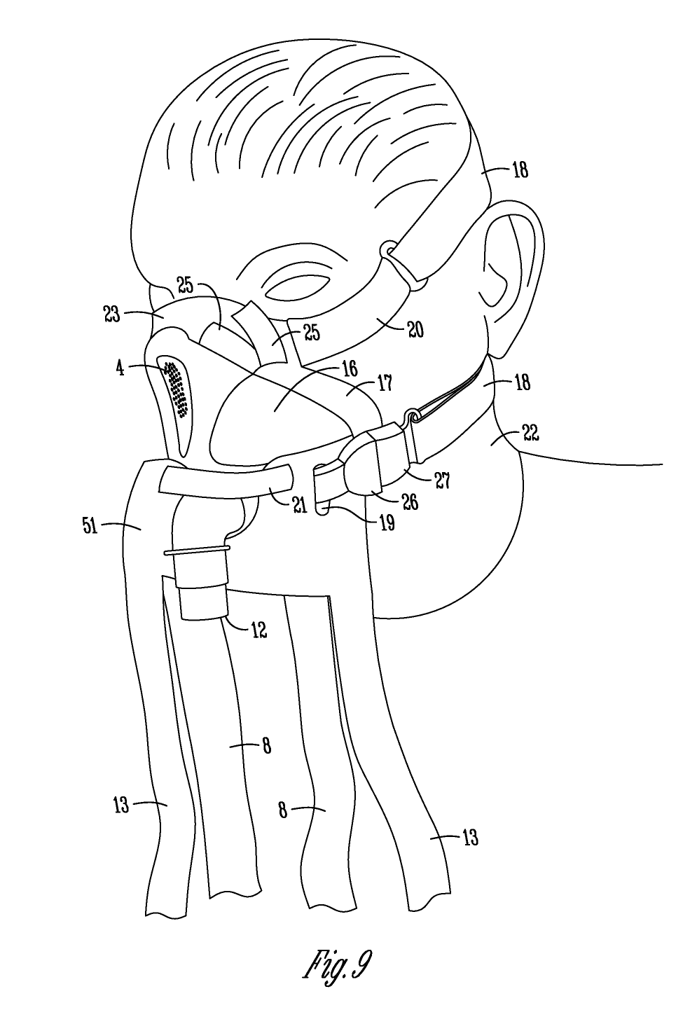

The housing-liner 30 according to an embodiment of the invention is first attached to the mask 10 as shown in FIG. 6, by pushing the female attaching means 26 through both holes 19 of the body 51 of the housing-liner, such that the smooth surface will oppose the skin of the user and the loop surface will be on the externalized side. Straps 14 are then wrapped tightly around the female attaching means 26 as shown in in FIG. 6 by Velcro means onto the body 51 of housing-liner 30, so that the housing-liner 30 is further tied or attached to the mask 10. It is also possible to attach the housing-liner 30 to mask 10 by only using the straps 14 wrapping around the female attaching means 26. Straps 14 also serve to tighten the chin support afforded by the body 51 of the housing-liner around the anterior-inferior surface of mandible, thereby creating more tightening pressure to keep the mandible from easily moving inferiorly and anteriorly from its juxtaposition with the maxilla; in essence keeping the user's mouth from opening widely. This is accomplished when the straps 14 are stretched and placed on top of each other with hook Velcro means onto exterior loop Velcro side of 51. In addition, the straps 14 enhance the close anatomical relationship of the maxilla with the mandible, and will minimize movement of the cushion 17 of the mask in contact with the patient's facial contour, and thereby minimize further air leaks.

It is also understood that the housing-liner 30 can be made without the addition of straps 14 to attach the housing-liner 30 to the mask 10. The method of attaching the housing-liner 30 to the mask may be suffice by just pushing the female attaching means 26 through both holes 19 of the body 51 of housing-liner 30. In addition, other attaching means may be employed based upon the design of the particular mask for which the system is designed.

Next the single strap 21 shown in FIGS. 5 wraps over the superior pole of the hose connector 12 and ties both sides of the housing-liner 30 at the superior edge of its body 51. This is shown in FIG. 6 as the strap 21 affixed over the hose connecting both sides of the housing-liner and forming a hole or an enclosed opening 53 through which the hose connect is disposed. By so doing the hose connector 12 is now incorporated within an enclosed opening of 53. The strap 21 depicted in FIGS. 5 and 6 utilizes a hook and loop Velcro, however, as one skilled in the art appreciates, the strap could be of other type or materials as previously mentioned. Strap 21 also serves as a positioning means for the housing-liner 30 onto the mask 10 although it is not a permanent attaching means as by itself the housing-liner 30 could slip off the hose connector 12. In addition, before strap 21 is attached to the other superior edge of body 51, the user can easily stretch the body 51 of the housing-liner 30 laterally to push the female attaching means 26 through both holes 19 of the body 51.

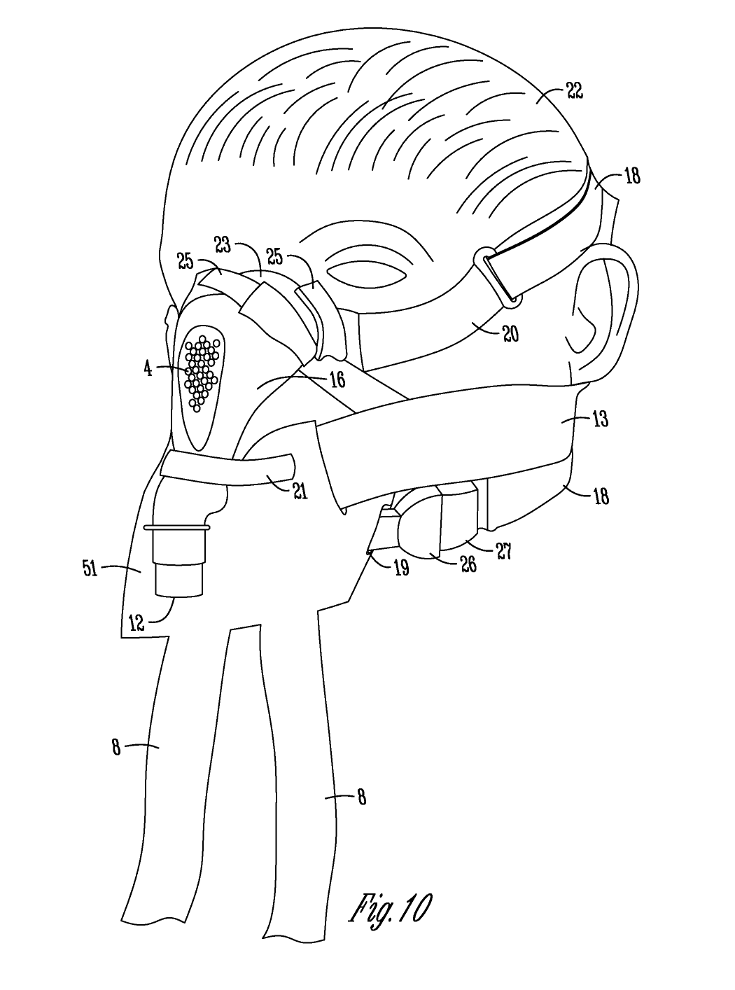

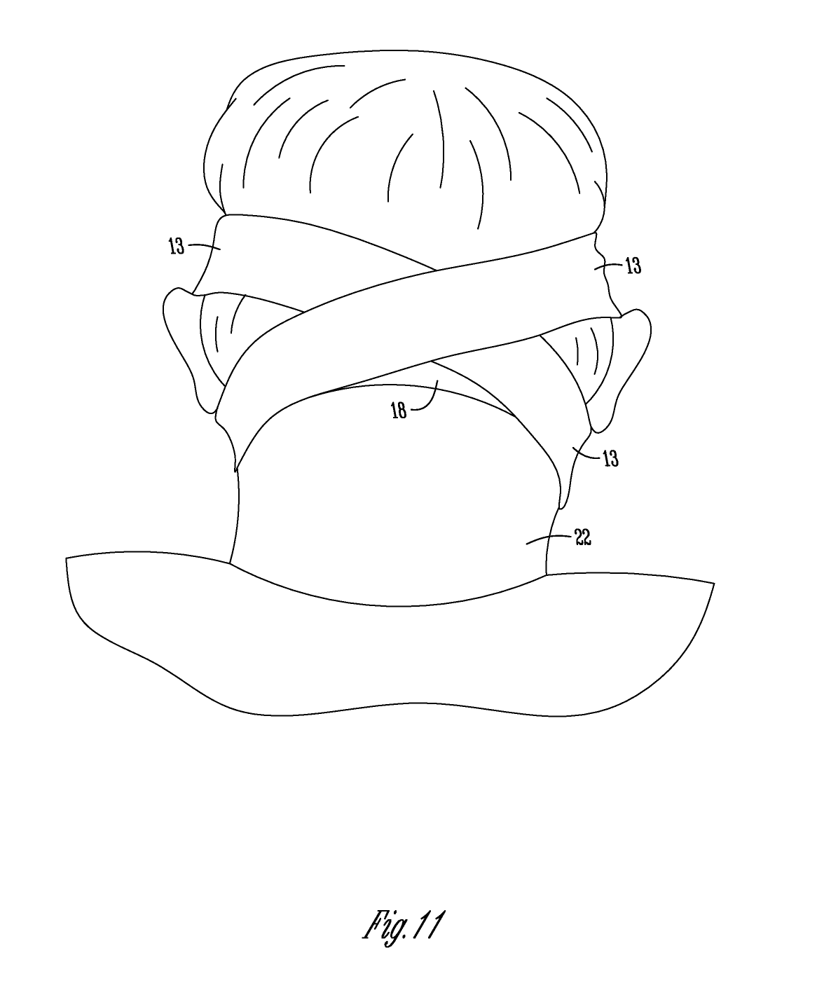

FIG. 7 illustrates an eye shield strap 23 which may be comprised of similar material as the central body and/or straps of the housing-liner. In an aspect, the eye shield strap 23 is made of stretchable thinly milled breathable material with a Velcro loop on one side and a smooth other side. This rectangular-like strip has two sets of identical side straps 25 situated at both shorter dimensional ends of the eye shield 23. The strap 23 is placed on the superior surface of the mask 10 by placing the strap 23 with the loop side up on the top surface of the mask at a location that is proximal and inferior to the user's eyes when the mask 10 is placed on the user's head. Straps 25 loop around posts 31 of the mask 10 (FIGS. 1 and 8) which join the upper headgear straps to the outer edge 16 of mask 10. By stretching the eye shield strap 23 and straps 25, good adherence will occur when the straps 25 comprised of Velcro hook are applied around the posts 31 and onto the top surface of strap 23 comprised of Velcro loop, thereby creating an attached eye shield designed to divert air leaks away from the user's eyes. It is also possible to invert the surface of the strap 23 inside the mask so that the loop or smooth surface interfaces between the user and the mask, and thereby minimize air leaks around the user's eyes.

Although eye shield strap 23 is separate in this embodiment shown in FIG. 8, the strap 23 could be integrated or attached to the housing-liner 30 of the invention in alternative embodiments by Velcro or other attaching means, so that the two parts can be manufactured as a single entity. Together the eye shield strap 23 and the housing-liner 30 is referred to as a system to prevent air leaks. The eye shield 23 can also be used on the mask 10 as a stand-alone element to minimize just air leaks onto user eyes.

Although the housing-liner 30 and the eye shield 23 are shown as separate and/or combined parts of a system to reduce air leaks, nevertheless the intention of the invention is such that either the housing-liner 30 and/or the eye shield liner 23 could be manufactured integrally as part of the mask 10 and/or its headgear. For example straps 13 could be integrated with the lower headgear straps to simplify manufacture of the invention, so that one or the other might by design be eliminated but the combination would provide some or all of the desired functions for both as described. These combinations can be accomplished by appropriate textile design and cut outs in combination with Velcro attaching means or other types of commonly used attaching means, such as sewing parts together, common hook and catch clips etc.

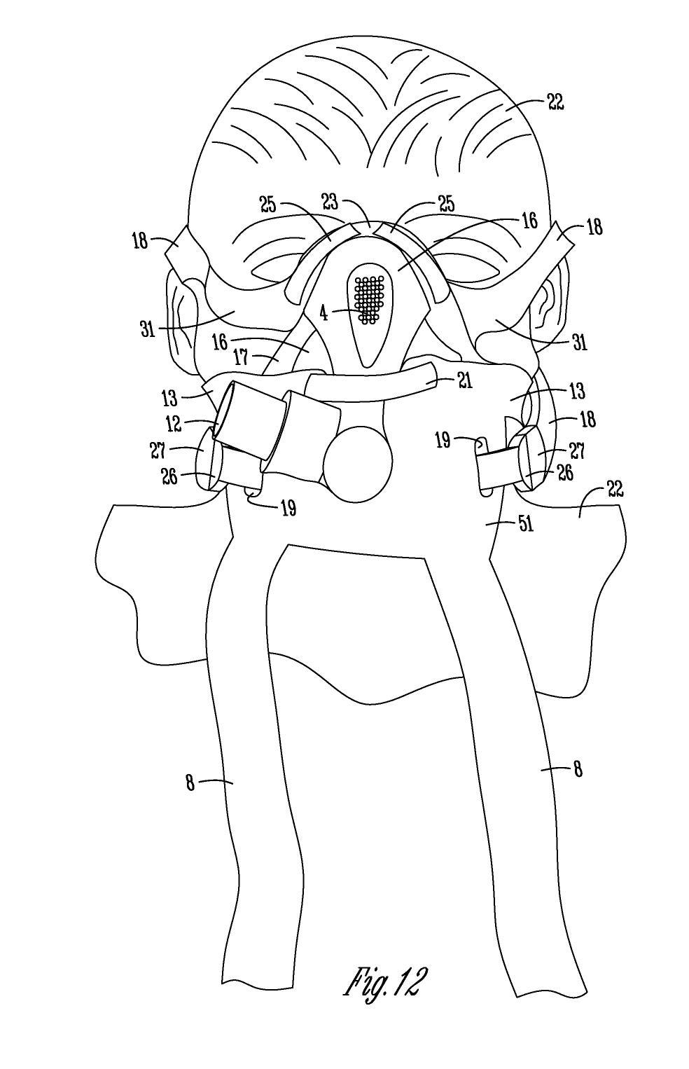

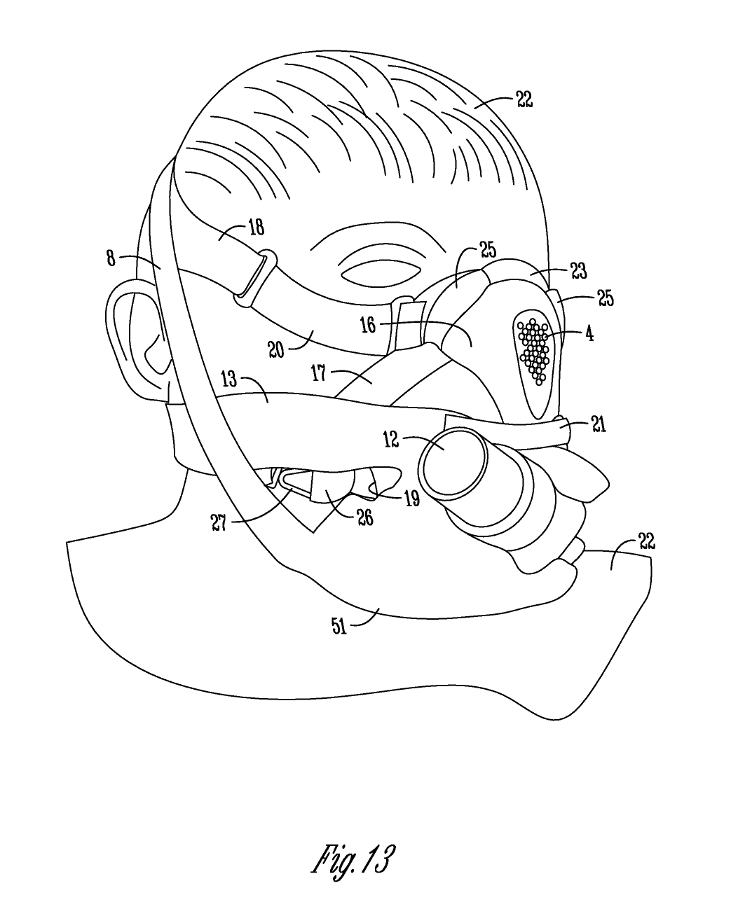

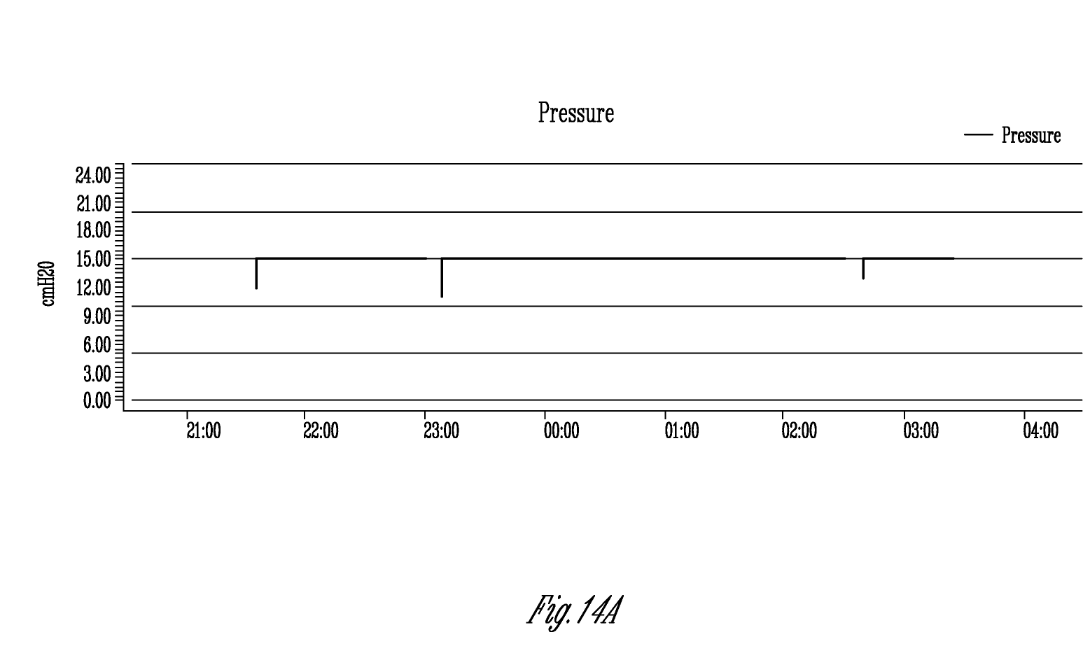

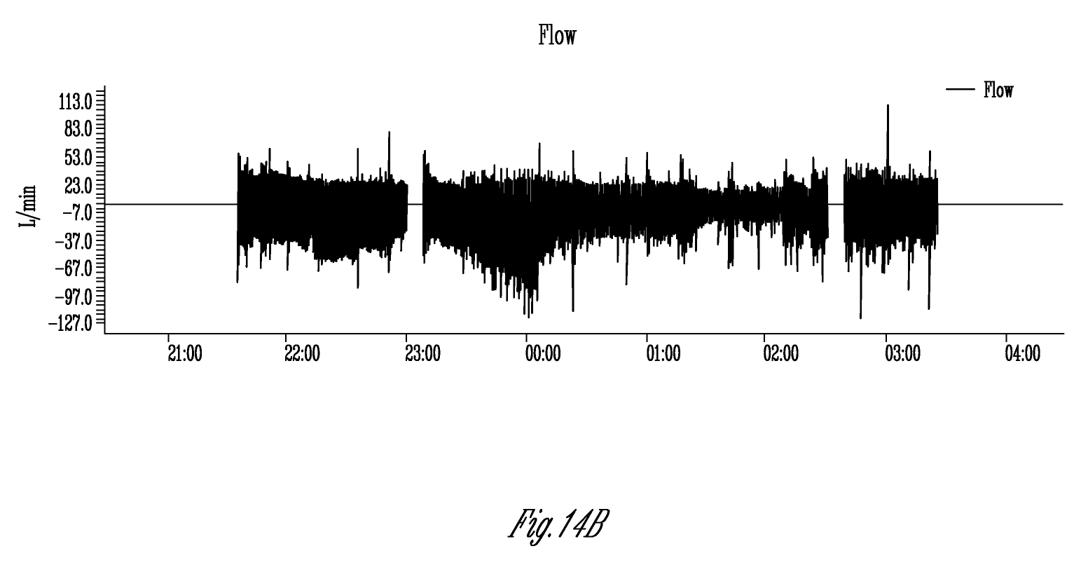

The housing-liner 30 according to an embodiment of the invention is further attached to the mask 10 by placing the housing-liner 30 attached to the mask 10 onto the user, as shown next in FIG. 9. The mask 10 with the housing-liner 30 and eye shield 23 is attached to the user's head 22, by placing the upper headgear straps 18 onto the user's head and then clasping the lower headgear attaching means (26, 27) on both sides. When the mask is placed on the user the eye shield 23 is optimally located at the closest junction of the nose (e.g. upper bridge of the nose close to the eyes) to the face to reduce air leaks around the eyes. The longer set of straps 13 of the housing-liner (as shown in FIG. 5) are then stretched around the lower part of the user's head beneath the ears, and then wrapped over each other, such that at the crossing of one over the other positions the straps. In an aspect, the crossing of one over the other with the hook and loop Velcro, further keeps them positioned from retracting backwards. This step of attaching straps 13 is shown in FIGS. 10 and 11.

In additional aspects, there can be pillows, not shown, which may also be referred to as bosses, but preferably referred to in this application as pillows, located reversibly or irreversibly on the surface of each strap 13 approximating the user's face. They can be located at any position but preferably when each strap 13 overlays the attaching means 26, 27, a point where there is often air leakage and the pillows serve as a means to create more moments of force with stretch to push inwardly at this location to minimize air leakage between the interface of the cushion 17 and the user's face. It should be understood that the placement of straps 13 around the user's head can be at other locations such as around the upper edge of the user's ears or in any other configuration to accomplish the functions as outlined.

According to the invention, the straps 13 have several functions: 1) Straps 13 allow the mask 10 with the attached housing-liner 30 to be firmly connected onto the user's head, and thereby maintain the interface positioning between the cushion 17 and face of user, especially when the user's head moves around in sleep, thereby reducing air leak; 2) Straps 13 allow adjustment of forces on the user's head, laterally and posteriorly, such that there is capability of flexibility of these forces for the user's comfort and to reduce air leaks; 3) Straps 13 provide inward pressure forces onto the lateral sides of mask 10, thereby reducing some air leaks and 4) the positioning of straps 13 on the lower jaw creates pressure over the inferior pole 40 (FIG. 1 of the mask 10). This fixes the position of the cushion 17 part of the inferior pole 40 onto the surface of the anterior chin of lower jaw, and thereby keeps the cushion from sliding upwards into the mouth of the user, which can awaken the user. In addition, slippage of the mask inside a user's mouth can cause a break in the air tight seal between the interface of the mask cushion 17 and the surface of user's chin.

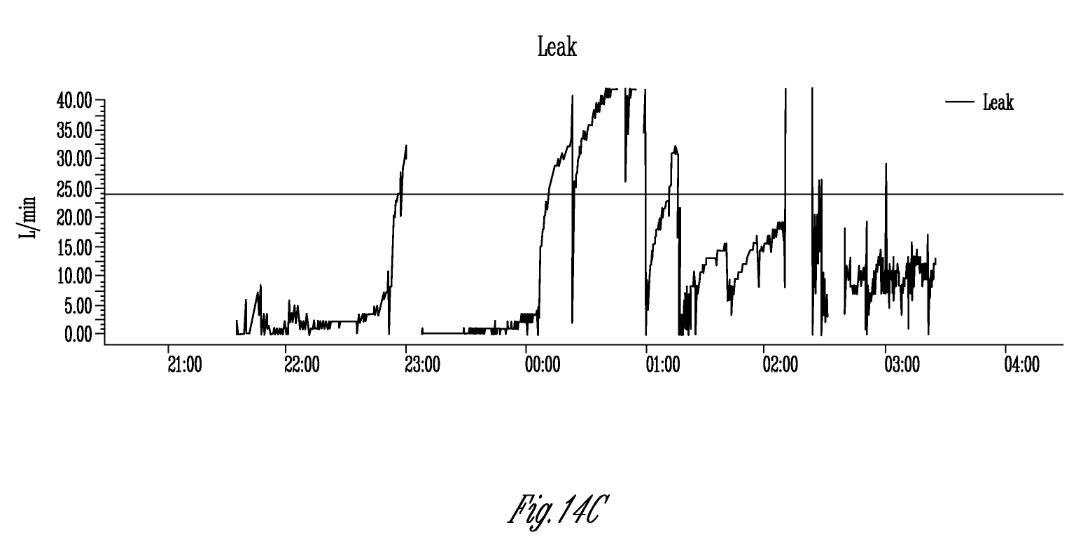

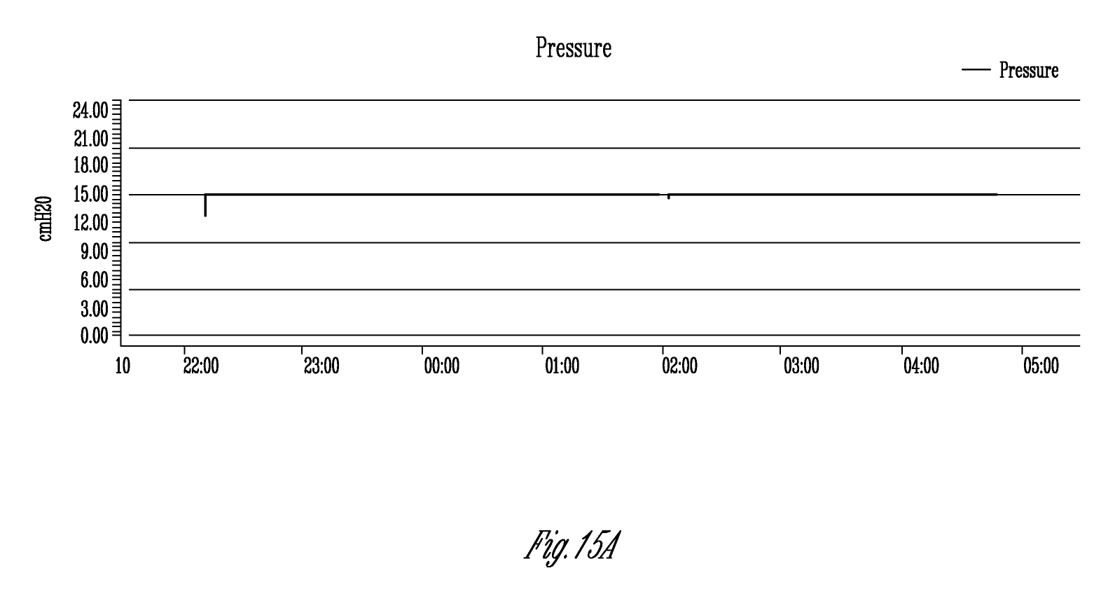

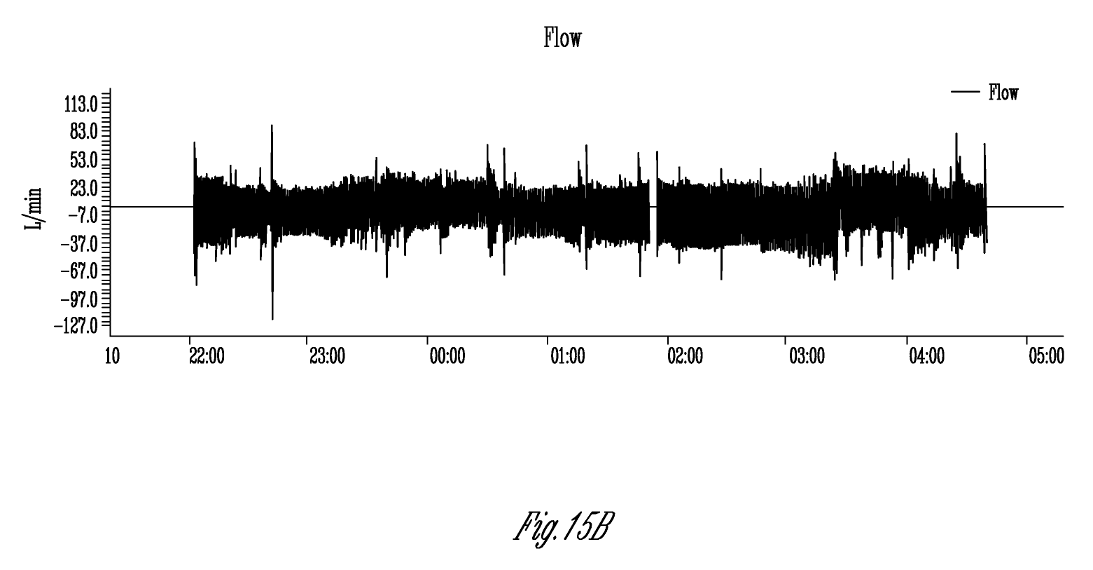

The final step for attachment of the housing-liner 30 to the mask 10 occurs when straps 8, as shown in an unattached fashion in FIG. 12, are then stretched under the surface of the chin to allow the body 51 of the housing-liner to become a chin support. In the embodiment of FIGS. 12 and 13, the first set of straps or strap 14 (if employed in a particular housing-liner system) is disposed beneath the straps of the system 8, 13. The straps 8 are then pulled upwards and with Velcro hook, the ends are attached to either the upper headgear straps which have Velcro loop or in combination with the ends of straps 13 which are also have Velcro loop. In some aspects, the attachment of straps 8 are placed relative to the horizontal line of the body 51 of the housing-liner from about 30° to about 90°, such as shown in FIG. 13. By placing the eye shield 23 closest to the junction of the nose and face, the straps 8 at these preferred angles provides forces towards the back and top of the user's head. This combination of forces will create a tighter seal between the housing-liner and the user's face to provide comfort and support as set forth according to the present invention.

Straps 8 have several functions: 1) they allow the chin support to be held in place so that the user's mouth remains almost closed inside the full face mask during sleep; 2) straps 8 apply pressure inwardly over straps 13 which in turn apply pressure inwardly on clasping attachments means 26, 27 of headgear straps and onto lateral borders of cushion 17, all helping to reduce air leaks; and 3) straps 8 also apply inward pressure over the lower pole 40 of mask (as shown in FIG. 1) to help fix it in place and prevent it from slipping up into the user's mouth. Straps 8 and 13 also apply inward pressure over the user's cheeks and thereby reduce the likelihood of puffing out of user's cheeks when air pressure builds up inside the user's respiratory tract. The straps 8 can each be placed anterior to the ears or posterior to the ears, and each can be positioned differently from the other or the same as the other. An exemplary positioning of straps 8 is shown in FIG. 13. Positioning is determined by the user to provide the best outcome for any or all of the following intended functions: patient comfort; improved securing of housing-liner 30 to patient's head; reduction of air leaks; slippage of the lower pole 40 of mask upwards into patient's mouth; and reduction of air cheek puffing.

In practice the eye shield is first applied to the superior portion of the mask 10 before the mask is used and where it can remain permanently fixed into position. Then the housing-liner 30 is attached to the mask 10 by pushing the female attaching means 26 through holes 19 in the chin support (e.g. central body) 51 of the housing-liner 30. Next the single strap 21 shown in FIGS. 5 wraps over the superior pole of the hose connector 12 and ties both sides of the housing-liner 30 at the superior edge of its body 51, creating an opening 53 to allow enclosure of the hose connector 12 into the housing-liner 30. Then the user optionally wraps straps 14 around the attaching means 26 and fixing both straps 14 to the exterior surface of 51 by Velcro or other attaching means. Now the entire system with the mask 10 is ready to place on the user as described in the prior paragraph. First the headgear attaching means 26, 27 interlock; then straps 13 are placed around the back of the head, followed by attaching straps 8 to headgear or ends of straps 13 by pulling them back and upwards to form chin support. To remove the system, straps 8 are pulled down, and straps 13 are unwrapped from the head. Then the user unclips the lower headgear attaching means (26, 27). In an emergency the latter can be done first and straps 13 removed to allow the user's mouth to open freely.

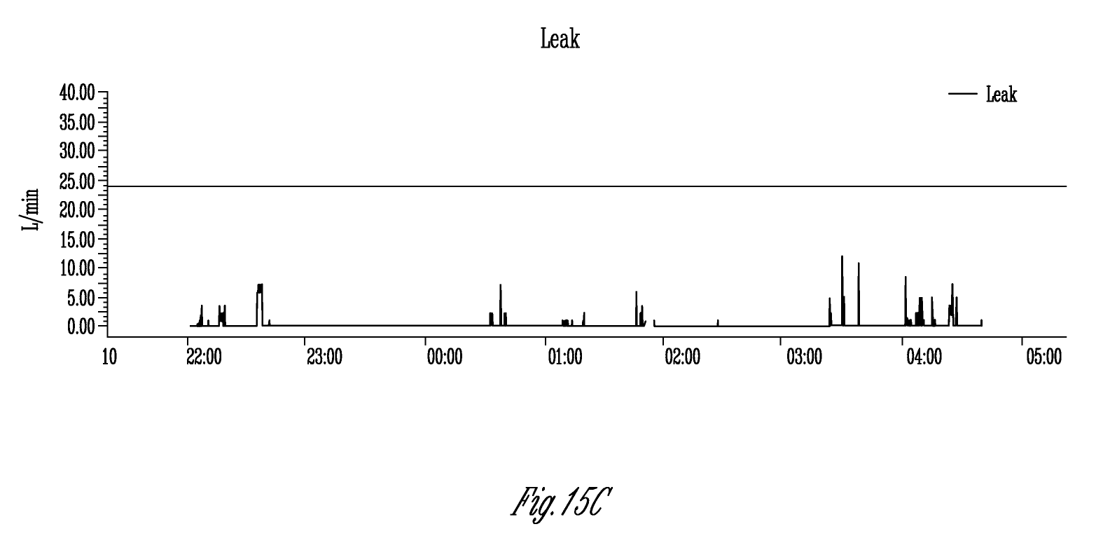

Functionally, when the system of the housing-liner 30 and the eye shield strap 23 are attached to mask 10, the objects of the proposed invention are accomplished. First, wearing the system reduces air leaks significantly as shown in FIGS. 14 and 15, in which air leaking was monitored with and without the use of the housing-liner 30 and eye shield 23 system for mask 10. In addition, the system muffles air leaks that may occur allowing for more peaceful sleep for the user and the sleep mate. The system with the eye shield 23 minimizes the annoying air leak directly on eyes that can create a dry eye condition with eye irritation, a condition frequently observed by eye specialists. Furthermore the system controls cheek puffing and prevents slippage of the inferior pole of mask into the user's mouth. Lastly and very importantly, the housing-liner provides a chin support, thereby obviating the need for a separate chin support strap system for individuals with a tendency to open their mouth during sleep. Hence all the designated objectives of this invention can be achieved.

The system can be modified for almost all known full face masks and also for nasal masks. A major modification can be made for the nasal CPAP mask with a chin support using longer straps 8 that will attach to the headgear by Velcro or other attaching means. The straps 13 can be modified to wrap around the user's face, preferably under the ears for placement. The eye shield 23 can be modified to fit each of these variant masks forms.

In some embodiments, the system may also be formulated to be permanently affixed to a CPAP mask. This may occur by a plurality of fixation mechanisms that may be employed by manufacturers of CPAP masks, as one skilled in the art will be able to ascertain. Beneficially, in such an aspect a user does not require all of the steps to affix a housing-liner and/or eye shield to a mask.

In addition, the system and/or housing-liner described in this invention for CPAP equipment may also be applicable to other types of masks, such as for protective masks used by firemen, pilots etc. This invention could improve sealing of the mask on the user and prevent air leakage from the environment into the user and from air pressure leaks when air compressors and/or oxygen are delivered. These and other embodiments of the invention are easily ascertained by one of skill in the art and are included within the scope of the present invention.

From the above discussion, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. In particular it is possible to parse the system to utilize each element in this invention individually or in varied combinations such as a chin support 51 integrated reversibly or irreversibly as part of a mask and/or its headgear.

Similarly an eye shield 23 with or without the housing-liner 30 could be utilized. Such modifications are intended to fall within the scope of the claims of this invention.

### EXAMPLE 1

A patient with sleep apnea was fitted with a standard CPAP mask (model shown in FIG. 1) to monitor the amount of air pressure, air flow and air leakage during a normal night's sleep. The results of use of a commercially-available CPAP mask are shown in FIG. 14, depicting the measured pressure, flow and air leakage experienced by user without the housing-liner 30 and eye shield 23 system according to the invention.

The same user was evaluated using an apparatus according to the invention, including the housing-liner and eye shield. FIG. 15 shows the comparative graphs of the same patient demonstrating a significant improvement in reduction of air leakage when wearing the same commercial CPAP mask as worn in FIG. 1 with a housing-liner 30 and eye shield 23 system according to the preferred embodiment of the invention. In addition, beneficially according to the invention, the reduction in air leakage further results in the clinical reduction of episodes of sleep apnea. In some aspects of use according to the invention, the system including the housing-liner with/without eye shields reduces sleep apnea in a patient.

1. A housing-liner apparatus for use with an airflow mask, comprising:
a body, wherein said body directly interfaces with an airflow mask, said mask comprising a cushion and at least one head-strap for attachment of said mask to a user, such that said cushion directly interfaces with user's face;
an attaching means either disposed within said body and/or connected to said body to provide at least one point of connection of said body to said mask; and
at least one or more straps attached to said body;
wherein the housing-liner reduces or eliminates air leakage from the mask and minimizes noise due to said air leakage.

2. The apparatus of claim 1, wherein the body of the housing-liner maintains a fixed interface relationship between said cushion and user's face during sleep.

3. The apparatus of claim 1, further comprising a chin support and/or at least a second strap affixed to said body of the apparatus, wherein said second strap wrap over said head-strap of said mask and around the user's head or wrap over both lateral sides of said mask and said user's head.

4. The apparatus of claim 1, wherein said mask further comprises an attaching means, wherein said mask attaching means comprises at least one strap attached to said body, wherein said strap wraps-around said mask attaching means providing further connection of said body to said mask and/or affixes two points on said body providing an enclosure for a hose connector of said mask.

5. The apparatus of claim 1, wherein said mask further comprises an attaching means, wherein said mask attaching means comprises at least one hole disposed within said body to accommodate said mask attaching means that is disposed through said hole allowing connection of said body to said mask.

6. The apparatus of claim 1, further comprising at least one additional strap that is irreversibly or reversibly attached to said body, wherein said additional strap provides chin support by reversibly attaching to said head-strap of said mask and/or to said apparatus.

7. The apparatus of claim 1, further comprising an eye shield, wherein said eye shield comprises a rectangular-shape having at least one strap, wherein said strap is affixed to a portion of said rectangular eye shield, and wherein said strap is reversibly or irreversibly attached to either a superior pole of said mask or to said portion of said housing-liner apparatus to divert air leaks away from the user's eyes.

8. The apparatus of claim 1, wherein the body and/or straps are made of a stretchable textile material.

9. The apparatus of claim 1, wherein the body and/or straps are made of a thermoplastic material.

10. The apparatus of claim 7, wherein the eye shield is made of a stretchable textile material and/or a thermoelastic plastic material.

11. A system for reducing air leaks from an airflow mask comprising:
an airflow mask comprising a cushion that directly interfaces with a user's face, a head-strap attaching means to connect said mask to said user, and an inlet for air and/or oxygen;
a source of air and/or oxygen delivered to said airflow mask; and
a housing-liner apparatus reversibly or irreversibly attached to and surrounding said mask, wherein said housing-liner comprises a body in direct contact with said airflow mask, at least one attaching means either disposed within said body and/or connected to said body to provide at least one point of connection between said body and said mask, and at least one strap attached to said body, wherein said strap wraps-around said head-strap attaching means and/or said user's head and/or chin providing further connection of said body to said mask;
wherein the housing-liner reduces or eliminates air leakage from the mask and minimizes noise due to said air leakage.

12. The system of claim 11, further comprising a chin support and/or an eye shield reversibly or irreversibly attached to said mask or to said housing-liner apparatus.

13. The system of claim 12, wherein said eye shield is attached to a superior pole of said mask.

14. The system of claim 11, wherein said housing-liner apparatus comprises at least one strap that wraps around the head-strap attaching means of said mask and/or the user's head, and/or that affixes two points on said body providing an enclosure for a hose connector of said mask.

15. The system of claim 11, wherein said housing-liner apparatus comprises at least one strap that wraps around both lateral sides of said mask and said user's head.

16. The system of claim 11, wherein said housing-liner apparatus comprises at least one strap that is irreversibly or reversibly attached to said body to provide chin support, wherein said strap reversibly attaches to said head-strap attaching means and/or said housing-liner apparatus.

17. The system of claim 11, wherein said housing-liner apparatus further comprises a pillow, wherein said pillow stretches said strap(s) to apply greater inward force onto said mask when said strap(s) is wrapped around said head-strap attaching means of said mask and/or around said user's head.

18. A method of improving the effectiveness of an airflow mask including reducing air leakage and/or noise associated with air leakage, comprising:
providing the housing-liner apparatus of claim 1 to a user in need of an airflow mask, wherein the housing-liner is reversibly and/or irreversibly fixed to said mask and maintains such fixed position while a user sleeps, wherein said housing-liner surrounds an outer edge of an airflow mask comprising a cushion that directly interfaces with said user's face, a head-strap attaching means to connect said mask to said user, and an inlet for air and/or oxygen;
positioning the mask and housing-liner apparatus around the user's nose and/or mouth;
providing a source of air and/or oxygen to said mask; and
reducing airflow between an interface of said cushion of said mask and said user's face.

19. The method of claim 18, further comprising reducing episodes of sleep apnea.

20. The method of claim 18, further comprising reversibly or irreversibly fastening an eye shield to said mask and/or said housing-liner apparatus.
